SEQUENCE LISTING (2 sequences)

SEQ ID NO: 1 - DNA, 34 bp, Artificial Sequence
  synthetic sequence
aaagaattcaccatgtccaaaaacaaggaccaac

SEQ ID NO: 2 - DNA, 27 bp, Artificial Sequence
  synthetic sequence
ttactcgagtactggcgtggtgtgttg

(19) United States
(12) Patent Application Publication
(10) Pub. No.: US 2020/0123235 A1
(43) Pub. Date: Apr. 23, 2020

(54) BIOMIMETIC POLYMERS FOR THE PREVENTION AND TREATMENT OF VIRAL DISEASES

(71) Applicant: RensaVir, Inc., Rockville, MD (US)

(72) Inventors: Mark T. Martin, Rockville (US); Anthony R. Rees, Uppsala (SE)

(21) Appl. No.: 16/733,734

(22) Filed: Jan. 3, 2020

Related U.S. Application Data
(62) Division of application No. 15/448,792, filed on Mar. 3, 2017, now abandoned.
(60) Provisional application No. 62/302,904, filed on Mar. 3, 2016.

(51) Int. Cl. C07K 16/10; G01N 33/569; A61M 1/34; A01N 25/00; G01N 33/68; C08F 222/10; C08F 220/28; C08F 220/06; A61L 2/23; A01N 37/18; A01N 37/06; A61M 1/36

(52) U.S. Cl. CPC C07K 16/1027; A61K 2039/505; A61M 1/3486; A01N 25/00; G01N 33/68; C08F 222/1006; C08F 220/28; C08F 220/06; C07K 16/10; A61M 1/34; A61L 2/23; A01N 37/18; A01N 37/06; A61M 1/3601; A61M 2202/206; G01N 2500/20; G01N 2500/04; G01N 2430/60; G01N 2333/135; G01N 2333/08; C08F 2222/1013; C08F 2220/281; C07K 2318/20; G01N 33/56983

(57) ABSTRACT

Antiviral biomimetic polymers (ABPs) are disclosed that can be used to prevent and/or treat viral disease. The ABPs are discovered by a process involving high-throughput screening of polymer libraries using disease-relevant bioactive molecules as target molecules. ABPs can be nanoscale (termed nanoABPs) or larger. Methods are described for the preparation and use of ABPs as prophylactics and therapeutics (in vivo) and as preventative agents, for example, in personal protective equipment (ex vivo). ABPs can be used to prevent and treat viral diseases including those caused by Filoviridae.

### CROSS REFERENCE TO RELATED APPLICATIONS

The presently disclosed subject matter claims the benefit of U.S. patent application Ser. No. 15/448,792, filed Mar. 3, 2017, which claims priority to U.S. Provisional Patent Application Ser. No. 62/302,904, filed Mar. 3, 2016; the disclosures of which are incorporated herein by reference in their entireties.

### TECHNICAL FIELD

Biomimetic polymers according to the presently disclosed subject matter are polymers that function as artificial antibodies to bind a selected biological target with high selectivity, affinity, and avidity. The presently disclosed subject matter relates to antiviral biomimetic polymers (ABPs). NanoABPs are a subset of ABPs that are nanoscale in size, i.e., on a similar scale to that of naturally occurring bio-macromolecules such as antibodies. In the presently disclosed subject matter, ABPs are used in vivo or extracorporeally as therapeutic agents against viral diseases or ex vivo as prophylactic agents against infection by viral disease vectors. The presently disclosed subject matter falls within the technical fields of medicine, virology, pathology, polymer chemistry, high throughput screening, biochemistry, protein chemistry, and molecular immunology.

### BACKGROUND

### Biomimetic Artificial Polymers

Man-made polymers often resemble natural proteins in that they can be three-dimensional polymeric structures containing functional groups capable of forming covalent or non-covalent interactions (including hydrogen bonds, charge interactions, hydrophobic interactions, ionic bonds, and van der Waals forces) with small or large molecules. Man-made polymers can be designed or empirically discovered to carry out particular binding functions. In addition, such polymers can be produced as either biostable and storage stable or as storage stable but biodegradable, the latter achieved by adventitious use of biodegradable building blocks (monomers).

One problem with natural antibodies is that they have limited shelf lives and must normally be stored in a refrigerated or frozen state until use. Biomimetic polymers, on the other hand, are stable and can be kept indefinitely at room temperature without loss of binding activity. This amplifies the time-dependent cost advantage of polymers since protein and nucleic acids must be discarded if not used during their shelf life. An aspect of stability is where the anti-viral agent is to be used in developing countries where refrigeration is often scarce and drug-costs must be kept low. For example, there is currently a severe AIDS epidemic in sub-Saharan Africa. In this part of the world temperatures are high and the medicine must often be administered in rural areas far from refrigeration. Added to that, the populations in such areas are normally extremely poor and unable to afford access to expensive medicines. Thus, synthetic polymers have advantages over antibodies as medicines. Similarly, it is impractical for cost and stability reasons to use natural antibodies as capture agents in personal protective equipment such as gloves and surgical masks, but low-cost, stable biomimetic synthetic polymers would be ideal for these types of applications.

### Molecular Imprinting

Molecular imprinting is one method of preparing biomimetic polymers, the product known as a molecularly imprinted polymer, or MIP. Molecular imprinting is a well-established area of science (Wulff & Sarhan (1972); Arshady & Mosbach (1981); Haupt & Mosbach, 2000; Ngo, 1993; Wulff, 1998; Shea, 1994). In molecular imprinting monomers are polymerized in the presence of a non-polymerizing “print molecule” which can interact non-covalently with the monomers or as a metastable covalent complex that can be broken post-polymerization. In non-covalent molecular imprinting, the most commonly used method, the polymer is believed to wholly or partially surround the print molecule in such a way that there is substantial shape and electronic complementarity between the polymer and the print molecule, much like the natural fit between an antibody and antigen. Research in the area has been ongoing for many years in many laboratories and numerous methods are well described in the literature. Molecular imprinting has been shown to be able to distinguish between even amino acids that are very similar in structure (glutamate vs. aspartate) and between D- and L-isomers of amino acids. A number of publications have shown that MIPs can be made to bind many small molecules, peptides, proteins and even nucleotides, nucleosides, and bases (Yoshikawa et al, 2001; Spivak et al., 1997; Spivak & Shea, 1998; Mathew & Buchardt, 1995; Mathew-Krotz & Shea, 1995; Shea et al., 1993).

MIPs can be made from a range of different monomers, most of which have high surface densities of some kind of functional group, such as a carboxyl group (for example, acrylates and methacrylates) and cross-linking monomers such as divinylbenzene. Typically, polymerization does not always completely utilize all vinyl groups in the cross-linking monomers leaving free reactive groups via which other functional groups can be introduced during a post-polymerization modification step.

### Non-Imprinted Polymers

During molecular imprinting it is normal to carry out a parallel polymerization in the absence of a print molecule to enable the imprinting effect to be quantified. These control polymers are known as non-imprinted polymers, or NIPs. NIPs show lower selectivity for the print molecule than MIPs when the finished polymers are compared in binding studies. To quantitate this, an imprinting factor, k′, is used (k′=k_{MIP}/k_{NIP}) where k′ is typically a distribution coefficient in chromatography or other separation method and which can have a value anywhere between 1 and 10 or higher.

A disadvantage of MIPs is that they require a print molecule or other molecular entity to form the complementary site. When using as an imprint a protein or even larger macroentity such as a virus or cell, large scale preparation of particles is complicated, costly and open to safety issues downstream since the print entity must be removed before the particles can be used. With regard to NIPs, few examples have been described where NIPs can be shown to have the same level of specificity as the corresponding MIP.

### Pathogenic Viruses

Viruses are small infectious agents that can only multiply within the cells of animals, plants, and bacteria. The structures of viruses are simple compared to living cells and contain a small haploid DNA or RNA genome and a protein or glycoprotein coat called a capsid. In addition, some viruses called enveloped viruses are surrounded by a lipid membrane. There are at least 5450 known species of viruses. Many viruses cause human diseases including but not limited to; influenza virus (flu), smallpox virus (smallpox), herpes simplex virus 1 (cold sores), Epstein Barr virus (cancer), human immunodeficiency virus (AIDS), human papilloma virus (warts, cervical cancer), norovirus (food sickness), hepatitis virus (hepatitis), and rhinovirus (common cold).

A number of viruses appear on the United States National Institutes of Allergy and Infectious Disease (NIAID) list of Emerging Diseases/Pathogens list, which are important as potential bioterrorism weapons. These are; Variola major (smallpox) and other related pox viruses, and viruses that cause viral hemorrhagic fever including Arenaviruses (Junin, Machupo, Guanarito, Chapare, Lass, and Lujo), Bunyaviruses (Hantaviruses causing Hanta Pulmonary Syndrome, Rift Valley Fever, and Crimean Congo Hemorrhagic Fever, and other hantaviruses), Flavaviruses (Dengue and Zika), and Filoviruses (Ebola and Marburg), Caliciviruses, Hepatitis A, West Nile virus (WNV), LaCrosse encephalitis (LCEV), California encephalitis, Venezuelan equine encephalitis (VEE), Eastern equine encephalitis (EEE), Western equine encephalitis (WEE), Japanese encephalitis virus (JE), St. Louis encephalitis virus (SLEV), Nipah and Hendra viruses, tickborne hemorrhagic fever viruses (Bunyaviruses including Severe Fever with Thrombopenia Syndrome virus and Heartland virus and Flaviviruses including Omsk Hemorrhagic Fever virus, Alkhurma virus, Usutu virus, and Kyasanur Forest virus), tickborne encephalitis complex flaviviruses (Tickborne encephalitis viruses, European subtype, Far Eastern subtype, Siberian subtype, Powassen/Deer Tick virus), yellow fever virus, influenza virus, rabies virus, Chikungunya virus, highly pathogenic coronaviruses including severe acute respiratory syndrome virus (SARS-CoV) and MERS-CoV, and Mayaro virus.

### In Vivo Anti-Viral Agents

Currently there are relatively few prophylactic or therapeutic agents for viral diseases. Those that exist fall into one of four categories: (1) specific virus inhibitors (e.g., HIV protease inhibitors, RNA interference (RNAi)), (2) vaccines, which in some cases, notably HIV and RSV, can be difficult to produce, (3) pro- or anti-inflammatories such as interferons (Rider et al., 2011), and (4) specific antibodies (e.g., Synagis/Palivizumab, Medimmune) for RSV). Specific antibody anti-viral therapeutics and vaccines are generally challenging because viruses often evolve rapidly to escape the adaptive immune response. In particular, viral surface glycoproteins can be highly variable, hence hindering the development of antibody-based anti-virals.

Antiviral nanoparticles are under development and in use, but for the most part they are not biomimetic particles (see Definitions, below). For example, they can be materials such as silver particles coated with a biomolecule (Orlowski et al., 2014) or biodegradable drug delivery vehicles (Chiodo et al., 2014). There has been some effort in the development of biomimetic nanoparticles as antitoxin agents. For example, Shea has developed molecularly imprinted nanoparticles for the in vivo inactivation of the bee venom peptide, melittin (Zeng et al., 2010; Hoshino et al., 2012).

### Ex Vivo Virus Removal

Virus processing and virus removal are procedures to inactivate or remove active viruses from blood, plasma, or serum outside of the body. This can be done by extracorporeal removal using methods such as nanofiltration or chromatography or by inactivation of viruses using temperature, chemical methods, or UV irradiation (Horowitz et al., 2004).

In some cases, viruses are removed from blood products in blood withdrawn from one patient to be given to another by transfusion. In other cases, a patient's blood is treated to remove viruses and to be returned to the same patient. Examples of extracorporeal blood treatment methods include double filtration plasmapheresis (DFPP), hemodialysis, and therapeutic apheresis (plasmapheresis, plateletpheresis, leukapheresis). Hepatitis C virus removal and eradication (VRAD/Cascadeflo EC, trademarks of Asahi Kasei Kuraray Medical) is an effective technique to eradicate hepatitis C virus from infected individuals. Aethlon Medical, Inc. (San Diego, Calif.) sells or is developing the Hemopurifier for filtration of viral pathogens including human immunodeficiency virus (HIV), hepatitis C virus (HCV), and Ebola virus.

Nanoparticles have been designed to bind viruses using the method of molecular imprinting (Bolisay et al., 2006; Bolisay et al., 2007; Jenik et al., 2009) and non-biomimetic, non-specific simple polymers have been found to bind viruses based on anionic overall charge (Sakudo & Onodera, 2012). However, biomimetic, non-imprinted polymers purpose-designed to bind particular viruses have not been previously disclosed.

Thus, in accordance with aspects of the presently disclosed subject matter, biomimetic non-imprinted nanoscale polymers are provided, which are useful in vivo as protective or therapeutic agents against viral infection. The presently disclosed biomimetic non-imprinted polymers are useful ex vivo in personal protective equipment, as prophylaxis against virus infection and as extracorporeal capture agents to remove viruses from the blood of an infected patient.

### SUMMARY

This summary lists several embodiments and objects of the presently disclosed subject matter, and in many cases lists variations and permutations of these embodiments and objects. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a non-imprinted biomimetic polymer that binds to a virus. In some embodiments, the consequence of said binding causes the prevention of virus infection. In some embodiments, the consequence of said binding causes the diminishment or cure of a viral disease. In some embodiments, the polymer has high avidity by the virtue of simultaneous binding to more than one viral capsid proteins or to more than one epitope on any viral capsid protein. In some embodiments a plurality of polymers is provided comprising at least a non-imprinted biomimetic polymer that binds to a virus.

In some embodiments, said polymer is obtained by high throughput screening of a polymer library. In some embodiments, said polymer is a nanoscale biomimetic polymer. In some embodiments, said polymer is a microscale biomimetic polymer.

In some embodiments, said virus is Ebola virus. In some embodiments, said virus is respiratory syncytial virus (RSV). In some embodiments, said polymer binds to the capsid glycoprotein of Ebola virus, GP1,2. In some embodiments, said polymer binds to Protein G or Protein F of RSV.

In some embodiments, said polymer comprises a plurality of polymers wherein at least one of the plurality of polymers binds to a virus.

In some embodiments, the presently disclosed subject matter provides a method of rendering a virus less infectious comprising: a. providing a biomimetic polymer that binds to a virus, optionally wherein providing the biomimetic polymer comprises identifying a biomimetic polymer that binds to a virus by performing high throughput screening of a polymer library against said virus or a protein component of said virus, b. formulating said virus-binding polymer into a nanoscale or microscale antiviral binding polymer, and c. introducing said nanoscale or microscale antiviral binding polymer as an admixture into an environment in contact with said virus, wherein said nanoscale or microscale antiviral binding polymer binds to and sequesters said virus, thus rendering said virus less infectious.

In some embodiments, said environment is the circulating bloodstream of a human.

In some embodiments, said virus is Ebola virus. In some embodiments, said virus is respiratory syncytial virus (RSV).

In some embodiments, said environment is personal protective equipment selected from the group comprising surgical or face masks, hoods, goggles, garments, and boots. In some embodiments, said environment is a liquid, suspension, or emulsion selected from the group comprising body lotions, face lotions, hand lotions, hand sanitizers, liquid soaps, aerosol sprays, and mist sprays. In some embodiments, said environment is medical waste selected from the group comprising body bags, disposable plastic waste bags, towels, waste water filters, and sponges. In some embodiments, said environment is a filter for extracorporeal removal of viruses from blood by double filtration plasmapheresis (DFPP), hemodialysis, or therapeutic apheresis. In some embodiments, the therapeutic apheresis is selected from plasmapheresis, plateletpheresis, and leukapheresis.

In some embodiments, the presently disclosed subject matter provides a method of identifying an antiviral biomimetic polymer (ABP) comprising: a. preparing a library of polymers, and b. detecting virus protein binding to a member of said polymer library by high throughput screening.

In some embodiments, polymers of said library are synthesized in situ on a microarray slide. In some embodiments, polymers of said library are synthesized and then printed on a microarray slide.

In some embodiments, said virus protein is a capsid protein. In some embodiments, said virus protein is in the natural state on the surface of a virus, displayed on the surface of a virus-like particle, or an isolated protein molecule. In some embodiments, binding of the virus protein to the ABP is detected by immunodetection of the bound protein. In some embodiments, binding of the virus protein to the ABP is detected by a chemical protein detection method. In some embodiments, the anti-virus biomimetic polymer is a non-imprinted biomimetic polymer.

In some embodiments, a method is provided of rendering a virus less infectious. In some embodiments, the method comprises formulating an anti-virus biomimetic polymer (ABP) into particles and, introducing said ABP particles into an environment in contact with said virus, wherein said particles bind to and sequester said virus, thus rendering said virus less infectious.

In some embodiments, said ABP particles are formulated to the nanoscale or microscale. In some embodiments, said environment is the circulating bloodstream of a human. In some embodiments, said virus is Ebola virus. In some embodiments, said virus is RSV. In some embodiments, said environment is personal protective equipment selected from the group consisting of surgical or face masks, hoods, goggles, garments, and boots. In some embodiments, said environment is a liquid, suspension, or emulsion selected from a group consisting of body lotions, face lotions, hand lotions, hand sanitizers, liquid soaps, aerosol sprays, and mist sprays. In some embodiments, said environment is medical waste selected from a group consisting of body bags, disposable plastic waste bags, towels, waste water filters, and sponges.

Accordingly, it is an object of the presently disclosed subject matter to provide non-imprinted antiviral biomimetic polymers, methods of identifying such polymers and methods of using the polymers to prevent or treat viral infection.

An object of the presently disclosed subject matter having been stated hereinabove, and which is achieved in whole or in part by the presently disclosed subject matter, other objects will become evident as the description proceeds herein below.

### BRIEF DESCRIPTION OF THE DRAWINGS

FIG. 1 is a schematic drawing showing the sizes and shapes of typical viruses and virus-like agents relative to the size of a typical bacterium (E. coli, 2000×1000 nanometers (nm)) and a human red blood cell (10,000 nm diameter). The viruses shown include: bacteriophage T4 (225 nm), rabies virus (170×70 nm), adenovirus (90 nm), rhinovirus (30 nm), bacteriophage M13 (800×10 nm), chlamydia elementary body (300 nm), bacteriophages f2, MS2 (24 nm), tobacco mosaic virus (250×18 nm), viroud (300×10 nm), poliovirus (30 nm), prion (200×20 nm), vaccinia virus (300×200×100 nm) and Ebola virus (970 nm). The scale bars at the bottom indicate 100, 500, and 1000 nm.

FIG. 2 is a schematic drawing showing (top) the chemical structures of exemplary functional monomers and (bottom) the chemical structures of exemplary cross-linkers that can be used to prepare libraries of the presently disclosed antiviral biomimetic polymers for screening for binding to viruses and virus capsid proteins.

FIG. 3 is a schematic drawing showing an exemplary process for the preparation and high throughput screening of anti-virus biomimetic polymers. At the top, solutions of monomer, cross-linker and initiator are pipetted sequentially on a microarray surface, such as a glass slide or coverslip to create an array. The array can then be exposed to a virus (e.g., the Ebola virus). Binding of the virus to particular array members can be detected, for example, by exposing the array to an anti-virus antibody that is linked to a fluorescent label. Fluorescence at particular points on the array indicate particular polymers that have binding ability for the virus.

FIG. 4 is (top) a photomicrograph of the Ebola virus and (bottom) a schematic drawing of a partial Ebola virus viron showing capsid glycoproteins (GP) projecting from the outer viral envelope.

FIG. 5 is (top) a photomicrograph of a respiratory syncytial virus (RSV) near a cell surface receptor and (bottom) a schematic drawing of RSV including a lipoprotein coat that contains lipoproteins including: fusion protein (F), capsid glycoprotein (G), and SH protein. The interior of the virus contains intracellular proteins including nucleoprotein (N), phosphoprotein (P), matrix protein (M) and “large” protein (L), which includes RNA polymerase catalytic units.

FIG. 6 is a set of images of microscope slides containing polymer arrays as described in Example 3. The Blank slide (left panel) is scanned at 635 nm for autofluorescence. The No-Phage Assay slide was taken through all steps of Example 3 EXCEPT without the presence of M13 phage particles (right panel). The Phage Assay slide was taken through all steps of Example 3 (center panel). The circled spots are those showing positive M13 phage binding.

### DETAILED DESCRIPTION

In addition to the many viral diseases that exist today, an estimated five new human infectious diseases arise every year, many of which are viral (Jones et al., 2008). The presently disclosed subject matter describes methods and compositions of matter that enable prevention or treatment of viral diseases using antiviral biomimetic polymers (ABPs). The ABPs are selected and optimized by high throughput screening of polymer libraries, for example in microarrays.

The ABPs according to the presently disclosed subject matter have a number of advantages over antibodies in binding viruses: (1) ABPs have high-valencies which complement the high number of identical surface proteins on virus particles, (2) synthetic polymers are more stable than antibodies and hence have longer shelf lives, and (3) synthetic ABPs can be discovered more easily, are manufactured more readily and are more scalable than antibodies.

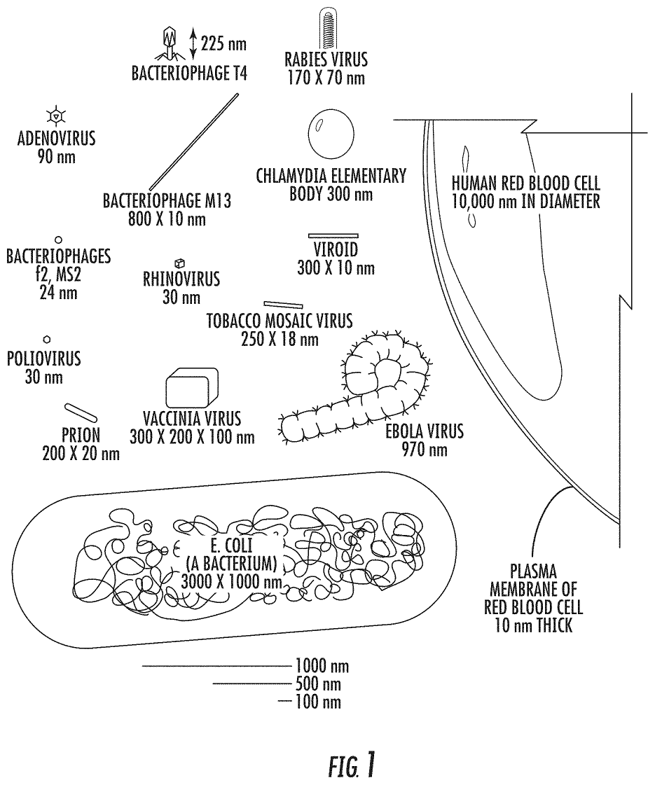

For practical anti-viral applications, ABP particles can be manufactured to be nanoscale (less than 1 micron in diameter) or microscale (equal or greater than 1 micron in diameter). ABPs can also be prepared in non-particle form such as a monolithic filter or membrane. NanoABPs are similar in size to viruses (FIG. 1) and preferred for in vivo uses. ABPs used outside the body can optimally be larger than nanoscale, depending on the application.

Virus-binding polymers (ABPs) have distinct advantages over bacterial or mammalian cell-binding polymers. In contrast to cells which are coated with hundreds of structurally diverse surface proteins, viruses are coated with only one or a very small number of proteins. If the structure of a viral surface protein is known, ABPs can be rationally designed. Moreover, because of the highly homogeneous virus protein landscape, ABPs will bind to multiple protein molecules of the same type, creating higher avidity and tighter binding than polymers empirically-discovered to bind to highly surface-heterogeneous cellular targets.

ABPs can be used as anti-viral therapeutic or protective agents. Nanoscale polymers (nanoABPs) can be administered to treat a virus-infected sick individual or to protect an individual who has recently been exposed or anticipates near future exposure to a particular virus.

ABPs can also be used outside of the human body to help prevent viral infection. Because they bind to a chosen virus, ABPs inhibit the entrance of that virus into the human body. Hence, infection prevention can be enhanced by including ABPs into personal protective equipment, soaps, wound protectants and other items. ABPs can also be used to eliminate virus from the blood or plasma of a patient in an extracorporeal manner, in which the blood or plasma is removed from a patient, filtered through ABP to remove virus, and returned to the patient. Uses outside of the human body can employ biomimetic polymers in formats larger than nanoscale.

### Abbreviations and Definitions

### Nonstandard Abbreviations:

AIBN, azobis-(isobutyronitrile); EDGMA, ethylene glycol dimethacrylate; Kd, dissociation constant; kDa, kilodalton; MAA, methacrylic acid; MIP, molecularly-imprinted polymer; ABP, anti-viral biomimetic polymer; nanoABP, nanoscale antiviral biomimetic polymer; NIP, non-imprinted polymer; RSV, respiratory syncytial virus; 2-Vpy, 2-vinylpyridine.

### Definitions and Abbreviations:

Antiviral Biomimetic Polymer (ABP): A virus-binding biomimetic polymer.
Artificial Antibody (or Receptor): A molecule that has one or more binding sites that is complementary in shape and/or charge to another molecule. The artificial biomolecule binds to the complementary molecule.
Biomimetic: The characteristic of possessing at least two different chemical functional groups, which is capable of forming a three-point complementary binding interaction with a biomolecule. As with natural biological binding interactions, functional biomimetic interactions typically have dissociation constants (Kd) of 100 micromolar or less, commonly less than 1 micromolar, and sometimes less than 1 nanomolar.
Biomimetic Substance: A non-biological substance possessing at least two different chemical functional groups, which is capable of forming a three-point complementary binding interaction with a biomolecule. Typical binding interactions include hydrogen bonds, charge interactions, hydrophobic interactions, ionic bonds, van der Waals forces, and covalent bonds.
EBOV: Zaire Ebola Virus.
Emulsion Polymerization: Polymerization whereby monomer(s), initiators, cross-linkers, and dispersion medium constitute an inhomogeneous system resulting in formation of particles or nanoparticles.
Functional monomer: A monomer used in polymer chemistry that upon polymerization retains a chemical moiety capable of forming an attractive interaction with another chemical moiety. Examples of attractive interactions include; hydrogen bonds, charge interactions, hydrophobic interactions, ionic bonds, van der Waals forces, and covalent bonds.
GP1,2: Knob-shaped capsid glycoprotein that coats EBOV.
Microscale: A scale larger than 1.0 micron and less than 1.0 cm.
Microscale Polymer (Fiber or Particle): A polymer (fiber or particle) with a cross-sectional radius of greater than 1.0 micron and less than 1.0 cm.
Molecular Imprinting: A process whereby specific binding sites to a chosen target (imprint) molecule are introduced into synthetic materials. The binding material is usually an organic polymer. Typically, functional and cross-linking monomers are co-polymerized in the presence of the imprint molecule, which acts as a molecular template. Subsequent removal of the template molecule reveals binding sites that are complementary in shape and size to the imprint molecule. In this way, molecular memory is introduced into the polymer, such that it has the ability to re-bind the imprint molecule with high specificity.
Nanoscale Antiviral Biomimetic Polymer (nanoABP): A virus-binding biomimetic polymer particle with a Stokes (hydrodynamic) radius of greater than 1.0 nm and less than 1.0 micron.
Print Molecule: A molecule to be molecularly imprinted, also known as a template molecule.
RSV: Respiratory Syncytial Virus.
RSV-G: RSV G protein-displaying virus-like particles.
VLP: Virus-like particle.

### Antiviral Biomimetic Polymers (ABPS)

Biological products, the active component usually being a protein, can be used as vaccines and drugs. The most attractive attribute of biologicals is the combination of exquisite specificity in their binding interactions with cellular molecules in vivo. Drawbacks associated with protein-based biologicals include difficulty in generation of an effective engineered product and high cost of manufacturing and quality control. Biologicals are produced from biological systems and can be contaminated with bacteria, fungi, mycoplasma, viruses, and residual host cell DNA and proteins. Manufactured protein products are inherently physically and chemically unstable, orally unavailable, and any adventitious immunogenicity can lead to side effects.

Because proteins are relatively unstable molecules, they are often not practical for many ex vivo uses, for example as anti-bacterial or anti-viral protectants in personal protective equipment.

The prevailing view in the art is that in order to obtain a degree of specificity for a particular target molecule in a polymer material, the target molecule must be used as an ‘imprint’ during the polymerization. In contrast to this prevailing view, the presently disclosed subject matter is based in part on assumptions regarding the similarity of antibody and polymer chemical diversity and the application of the theory of fitness landscapes, such as has been applied to antibodies by Macken & Perelson (1989, to polymer landscapes. Thus, in one aspect, the presently disclosed subject matter is related to the construction of a polymer library containing a large diversity of chemical functional groups representing binding sites with random shapes. Without being bound by any theory it is believed that if the ABPs lie scattered at random in the shape space and each ABP has the same accessible recognition volume, Vₑ, then the total volume of shape space covered by all the ABPs in the library is N_{ABP}.Ve. If the total available volume of shape space is V then each epitope would be recognized by N_{ABP}.Vₑ/V different ABPs. The probability that an epitope is not recognized by some ABP in the library repertoire is then:

P=exp (−N_{ABP}.Vₑ/V)

If the probability p(K) that a given ABP recognizes a random epitope with an affinity above some threshold, such as 1 in 10⁴ library members, then

P(K)=Vₑ/V≈10⁻⁴

From this the probability that an epitope will NOT contain an ABP in a library of 10⁴ members that recognizes it with an affinity above the threshold is then:

$$\begin{matrix}P = exp - ( {10}^{4} \cdot {10}^{- 4} ) \\ = exp ( - 1 ) = 0.37\end{matrix}$$

This says that if a library of 10⁴ polymers is constructed that contains chemical groups ‘fit’ for recognition of protein epitopes, such as in a viral coat protein, 37% of the epitopes will escape detection. This is a believed to be a reasonable situation since the presently disclosed subject matter includes the possibility to enlarge the library to as many as 10⁵ ABPs if necessary in which case P≈0 and all epitopes would potentially have an ABP with complementarity. By contrast, in the situation where only a small number of library members are present (as in, for example, a typical MIP experiment where around 100 polymers would represent the library), then

$$\begin{matrix}P = exp - ( {10}^{4} \cdot {10}^{- 2} ) \\ = exp ( - {10}^{2} )\end{matrix}$$

This number is so large that all epitopes would escape detection, hence the requirement for a print molecule at low library sizes.

Thus, in accordance with an aspect of the presently disclosed subject matter, imprinting is unnecessary if the polymer repertoire is large enough and diverse enough. Typically, MIP discovery is carried out at much lower repertoire sizes (for example in 96-well plates; Schillinger et al. (2012) where a screening library is described as 36 MIPs and 36 NIPs) since a print molecule ‘directs’ the polymer binding site. In the presently disclosed subject matter, by expanding the repertoire of polymer chemistries by several orders of magnitude, the imprint effect can be mimicked as a result of random combinations of monomer chemistries and stoichiometries. Thus, ABPs against a multitude of protein epitopes can be discovered without the use of a print molecule. This is advantageous from both a technical and economic point of view when contemplating the necessity for large scale production of ABPs, as envisaged in this application.

Preparation of ABPs

ABPs comprise polymers and can be prepared from monomers and cross-linkers using chemistry well known in the art. ABPs can be prepared individually, for example in a test tube or as collections or libraries of unique polymers, for example as spots on microarray slides or in the wells of microtiter plates. Designed large libraries of from 1000 up to 10,000 distinct polymers are attractive for high-throughput discovery of ABPs that bind to a particular target molecule.

### 1) Descriptive Summary of ABP Components:

a) Target Molecules

Target molecules can be particular biomolecules such as proteins, peptides, or carbohydrates found on the surface of a virus. The whole native virus displaying the target molecule can be used during polymer screening, discovery, and development. Alternatively, during polymer discovery and optimization, the target molecule can be displayed on the surface of a different, non-pathogenic virus or on a virus like particle (VLP). Another alternative is to develop polymers using the target molecule or a fragment thereof in a purified, isolated state in the absence of virus particles.

b) Monomers, Cross-Linkers, Initiators, and Porogens

One aspect of ABP technology is the discovery of virus binding polymers through high throughput screening of large combinatorial libraries of polymers. The libraries are chemically diverse and not limited to one type of monomer, polymer, or chemistry. However, for practical purposes, libraries are designed to include functional monomers and cross-linkers that are selected based on their potential for chemical complementarity to a chosen target molecule.

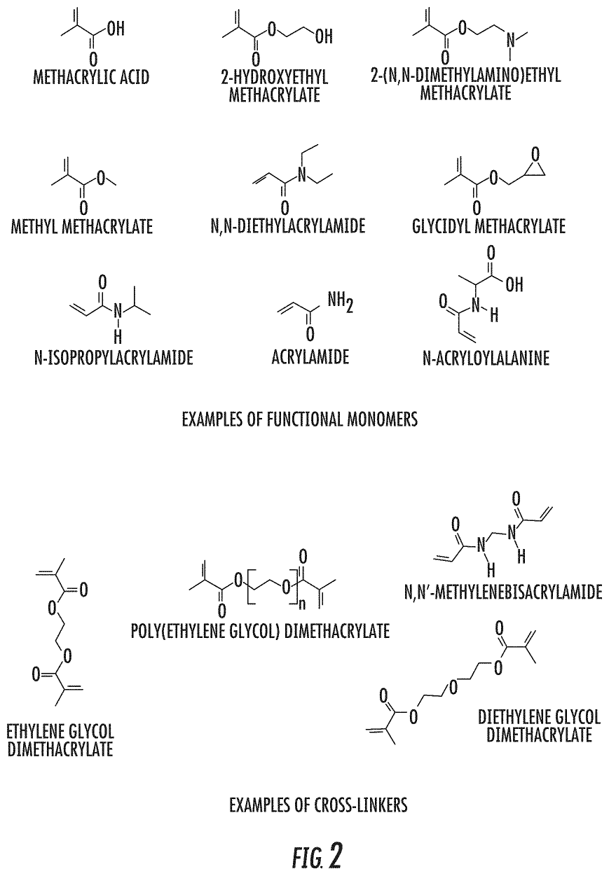

Subsets of typical monomers and cross-linkers are shown in FIG. 2. There are many commercially available monomers and cross-linkers and others whose synthesis is described in the literature. A non-exhaustive list of monomers includes acrylate (AA), methacrylate (MAA), ethyl acrylate (EAA), methyl methacrylate (MMA), hydroxypropyl methacrylate (HPMA), acrylamide (AM), methacrylamide (MAM), N,N-diethylacrylamide, N-isopropylacrylamide, glycidyl methacrylate, 2-hydroxyethyl methacrylate (HEMA), 2-(N,N-dimethylamino)ethyl-methacrylate, N-acyloylalanine, allylamine, N-vinylimidazole, N-vinylpyrrolidone, vinylpyridine, N-methacryloylhistidine, [2-(acryloyloxy)ethyl)trimethylammonium chloride (AEtMA-Cl), 2-(dimethylamino)ethyl methacrylate (DMAEMA), 2-(diethylamino)ethyl acrylate (DEAEA), N-(1,1-dimethyl-3-oxobutyl)acrylamide (DMOBAA), and N-isopropylacrylamide (NIPAA).

A non-exhaustive list of cross-linkers includes ethylene glycol diacrylate, ethylene glycol dimethacrylate (EGDMA), di(ethylene glycol) dimethacrylate, tri(ethylene glycol) dimethacrylate, tetra(ethylene glycol) diacrylate, poly(ethylene glycol) dimethacrylate, poly(ethylene glycol) diacrylate, ethylene dimethacrylate (EDMA), N,N′-ethylenebisacrylamide (DBA), divinylbenzene (DVB), 1,4-bis(acryloyl)piperazine (BAP), N,O-bismethacryloyl ethanolamine (NOBE), N,N′-methylenebisacrylamide (MBA), glycerol dimethacrylate, and (0,0-Bis(3-aminopropyl)polyethylene glycol) diacrylamide.

In addition, there are a number of polymerization initiation methods known in the art. Some chemical initiators and catalysts known in the art include, but are not limited to, ammonium persulfate, tetramethylethylenediamine (TEMED), 2,2′-azobis(2-isobutyronitrile) (AIBN), and 2,2′-azobis(2,4-dimethylvaleronitrile) (ABVN). Photoinitiation is also possible, for example, using 1-hydroxycyclohexyl phenyl ketone.

It is well known in the art that inert solvents known as porogens can be included in polymerization reactions where a viable pore structure in the particle is required. After polymerization, the porogenic solvents are removed by vacuum, displacement, or evaporation to create voids which are the basis of polymer porosity. Some typical porogens are toluene, acetonitrile, chloroform, tetrahydrofuran, and dimethylformamide.

In some embodiments, the presently disclosed subject matter provides a non-imprinted biomimetic polymer that binds to a virus, i.e., an antiviral, non-imprinted biomimetic particle. In some embodiments, the antiviral non-imprinted biomimetic polymer that binds to a virus is an acrylic polymer or a polyvinyl polymer. Said polymers can be homopolymers or copolymers. For example, in some embodiments, the polymer is a copolymer of two or more of the monomers listed above. In some embodiments, the biomimetic polymer is a crosslinked acrylic or polyvinyl polymer. However, while this disclosure primarily describes the use of acrylate and vinyl-based polymers, any other suitable polymer can be used. For example, other suitable polymers that can be prepared by one skilled in the art and that can be used as antiviral, non-imprinted biomimetic include, but are not limited to polyurethane/polyol polymers, such as described in Pernagallo et al. (2011) and Duffy et al. (2014b).

In some embodiments, the antiviral, non-imprinted biomimetic polymer that binds to a virus has a ‘apparent’ Kd for the virus (or a portion thereof, such as a surface protein thereof) of 100 micromolar (μM) or less, 50 μM or less, 10 μM or less, 1 μM or less, 500 nanomolar (nM) or less, 250 nM or less, 100 nM or less, 50 nM or less, or 1 nM or less. Binding of the polymer to the virus (or a portion thereof) prevents infection with the virus. In some embodiments, the virus is selected from the group comprising, but not limited to, influenza virus, smallpox or another pox virus, herpes simplex virus 1, Epstein Barr virus, human immunodeficiency virus, human papilloma virus, norovirus, hepatitis virus, rhinovirus, an Arenavirus, a Bunyavirus, a Flavavirus, a Filovirus, a Calcivirus, Hepatitus A virus, West Nile virus, an encephalitis virus, Nipah or Hendra virus, a tickborne hemorrhagic fever virus, a tickborne encephalitis complex flavivirus, yellow fever virus, rabies virus, Chikyngunya virus, Zika virus, Ebola virus, a coronavirus, severe acute respiratory syndrome virus (SARS-CoV), MERS-CoV, RSV, and Mayaro virus.

In some embodiments, the polymer binds to a capsid glycoprotein or lipoprotein of the virus. In some embodiments, the virus is RSV or Ebola. In some embodiments, the polymer binds to the capsid glycoprotein of EVOV, i.e., GP1,2. In some embodiments, the polymer binds to protein G or protein F of RSV. In some embodiments, binding of the polymer to the virus diminishes viral loading and/or symptoms of the virus infection in an infected individual or cures the infection.

In some embodiments, the antiviral, non-imprinted biomimetic polymer is a nanoscale biomimetic polymer (i.e., comprises a polymer particle having a cross-sectional radius or diameter of greater than about 1 nm and less than about 1 μm (e.g., about 2, 5, 10, 25, 50, 75, 100, 200, 300, 400, 500, 600, 700, 800, 900, or 999 nm)). In some embodiments, the antiviral, non-imprinted biomimetic polymer is a microscale biomimetic polymer (i.e., comprises a polymer particle with a cross-sectional radius or diameter of greater than about 1 μm and less than about 1 cm (e.g., about 1.5, 5, 10, 25, 50, 75, 100, 200, 300, 400, 500, 600, 700, 800, 900, 1000, 2000, 3000, 4000, 5000, 6000, 7000, 8000, 9000, or 9999 μm)).

### 2) Preparation of Polymers

a) Library Preparation

As described above, there are many potential polymers that can be made using combinations of chosen monomers and cross-linkers. A large number of combinations can be made from single monomer/single cross-linker combinations. For example, if one of three different cross-linkers is combinatorially combined with 20 singly used functional monomers, 60 different polymers (3×20) can be made. This size of library will have limited chemical diversity. Diversity can be further increased by mixing two or more monomers in a single polymerization reaction to yield an even greater diversity of composite polymers. For example, if any two of 20 functional monomers and any one of three cross-linkers are combinatorially combined, a possible 570 polymers can be made 3× (20!/(2!×18!)). Yet another dimension of diversity can be included by varying the ratios of individual monomers and cross-linkers. For example, if pairs of 20 functional group monomers and one of three cross-linkers are combined in 4 different ratios of concentrations (e.g., 3:1:1, 1:3:1, 1:3:1, and 1:1:1), then a total of 2280 polymers are possible (570×4). It is preferred to spot each polymer species in duplicate, triplicate, or quadruplicate.

For practical reasons it is preferred to prepare and screen polymer libraries on a small scale, ideally on microscale arrays. Polymer arrays can be prepared in microtiter plates (e.g., 96-, 384-, or 1536-well) or on microarrays (e.g., microscope slides or coverslips).

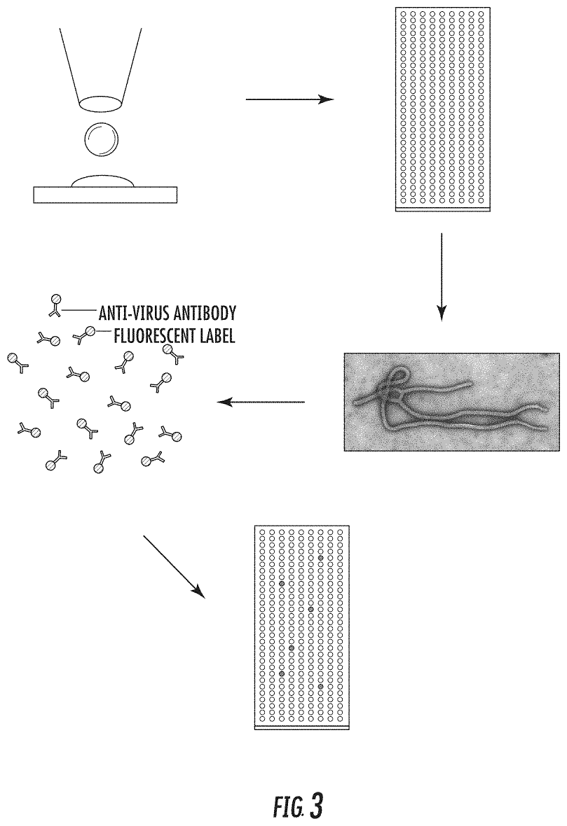

Polymers for screening can be prepared in advance and spotted, for example on a glass microscope slide. In those cases, pre-formed polymers can be spotted using commercial microarrayers (e.g., ArrayIt Corporation (Sunnyvale, Calif.); Core Life Sciences, Inc. (Liguna Niguel, Calif.)). Examples of contact printing of pre-formed polymers include Duffy et al (2014b) and Khan et al (2013). Alternatively, polymers can be prepared in situ on the slide by sequentially spotting individual or mixed monomers, cross-linkers, and initiators. For example, solutions of monomers, cross-linkers and initiators can be can be spotted sequentially on a microscope slide or coverslip to create an array as shown at the top of FIG. 3. Non-contact spotting can be performed using inkjet or laser printing microarray technology (Microdrop Technologies GmbH, Germany; Liberski et al., 2009a). Examples of polymer array synthesis by non-contact inkjet printing include Duffy et al. (2014a), Hansen et al. (2014), Liberski et al., (2009a), Liberski et al. (2009b), Zhang et al. (2008), and Zhang et al. (2013). Polymer arrays can also be prepared by hand simply by using standard adjustable volume pipetting devices.

In some embodiments, the antiviral non-imprinted biomimetic polymer comprises a plurality of polymers wherein the plurality of polymers comprises at least one polymer that binds to a particular virus or viral protein of interest. Thus, in some embodiments, the polymer is a mixture of polymers. In some embodiments, the polymer is a mixture of polymers present in a spot on an array that contains two, three, four, five, six, or more polymers. In some embodiments, the polymer is a combinatorial polymer library or subset thereof.

b) Screening and Detection.

Polymer arrays are screened for their ability to bind to the target molecule or target virus particle. The target is dissolved in a solvent, which typically resembles the solvent to be used in the final application such as an aqueous neutral buffer, human blood or serum. The concentration of the target molecule during screening is not critical. In some embodiments, the concentration of the target molecule should be high enough to be detectable while bound to polymer yet as close as possible to the concentrations expected to be encountered in the final application. Detection of the target bound to the polymer can be carried out by any method that does not interfere with the binding process. Methods can be immunological in nature, by using an anti-target antibody. The anti-target antibody can be labeled with a detectable moiety such as a colored or fluorescent tag as shown in FIG. 3 or it can be labeled with an affinity handle such as biotin, which can bind in a second step to streptavidin labeled with fluorescent tags or a color- or luminescence-generating enzyme such as horseradish peroxidase. Alternatively, detection methods can be non-immunological. Before screening, the target can be chemically modified with a radiolabel such as tritium, by a small detectable molecule, such as a fluorophore, or by a handle such as biotin, which can bind in a second step to fluorescently-labeled or enzyme-labeled streptavidin. Another non-immunological detection method is to detect polymer bound native target using a protein detection reagent such as silver stain.

Described below are exemplary methods for detecting binding of Ebola-like virus-like particles (VLP) (Wool-Lewis & Bates, 1998; Warfield et al., 2003; Watanabe et al., 2004; Ou et al., 2012) to an ABP by three methods: (a) direct colorimetric detection using silver stain, (b) direct fluorescent detection of prior fluorescein-labeled VLP, and (3) immunodetection using anti-VLP antibody, biotinylated secondary antibody, and fluorescently labeled streptavidin.

For silver stain protein detection, a commercial silver stain kit (Bio-Rad or Pierce) is used according to manufacturer's instructions. Colored spots indicating the presence of VLP bound to polymer are detected by digital photography or scanning.

For direct fluorescence detection of bound VLP, the VLP is labeled with a fluorescent molecule prior to the experiment. Common reactive groups include amine-reactive isothiocyanate derivatives including FITC, amine-reactive succinimidyl esters such as NHS-fluorescein or NHS-rhodamine, and sulfhydryl-reactive maleimide-activated fluors such as fluorescein-5-maleimide. Fluorescent conjugation is performed according to manufacturer's instructions. For example, Invitrogen Molecular Probes FluoReporter FITC Protein Labeling Kit (Cat. F6434) can be used. FITC, fluorescein isothiocyanate, is commonly used and can be lightly conjugated to avoid interfering with binding. The minimal degree of conjugation can be determined in advance by one skilled in the art. Detection of fluorescein-VLP bound to polymer can be determined using a fluorescence scanner (e.g., Axon GenePix Personal 4100A) using excitation at 494 nm and emission at 518 nm.

For immunodetection of polymer ABP-bound Ebola VLP, there are various anti-GP antibodies and Ebola ELISA kits that are commercially available (e.g., Alpha Diagnostic Intl., Inc., San Antonio, Tex.). Because it is not clear which kit or antibody will perform best with Ebola VLPs, a small number of kits can be purchased and screened on VLPs in an ELISA 96-well plate format in advance. One example is to use a rapid ELISA kit from Alpha Diagnostic Intl. (Human Anti-Zaire Ebola virus glycoprotein (GP) antibody rapid test, visual results in 10 min) to detect Ebola VLP bound to polymer. Another example is to use an anti-GP antibody such as purchased from Alpha Diagnostic Intl. (e.g., an ELISA kit) and to substitute the colorimetric enzyme in the kit with fluorescently-labeled streptavidin (Alexa Fluor 488 conjugated streptavidin, Life Technologies, Cat. #S-11223). Physical detection is performed with a photo scanner (colorimetric tests) or a fluorescence scanner (e.g., Axon GenePix Personal 4100A).

Accordingly, in some embodiments, the presently disclosed subject matter provides a method of preparing and/or identifying an ABP, wherein the method comprises: preparing a library of polymers and detecting virus and/or viral protein binding to members of said polymer library. In some embodiments, the detecting comprises performing high throughput screening of the polymer library (e.g., wherein the polymer library is provided for detection in an array format). In some embodiments, the polymers of the library are synthesized in situ on a microarray slide or in another array format (e.g., via the spotting of monomers, crosslinkers, and/or initiators onto the slide). In some embodiments, the polymers of the library are synthesized and then spotted or printed onto a microarray slide or other array format.

The presently disclosed subject matter has the advantage that it is versatile and can be used to produce ABPs against virtually any particular virus (i.e., against any virus target). There are many viruses that are difficult to raise antibodies against by conventional technologies. However, using the presently disclosed method, ABPs can be made and identified that bind to virtually any chosen target viral capsid molecule, and thus to any virus bearing a capsid coat or accessible surface protein. In some embodiments, the virus protein is a capsid protein. In some embodiments, the virus protein is present on the surface of the virus (i.e., in its natural state on the surface of a virus). In some embodiments, the virus protein is displayed on the surface of a virus-like particle. In some embodiments, the virus protein is an isolated protein molecule.

In some embodiments, the binding of the virus protein to the ABP is detected by immunodetection of the bound protein (e.g., via the binding of an anti-protein antibody labeled with a fluorescent label or with an enzyme that catalyzes the formation of a fluorescent molecule). In some embodiments, the binding of the virus protein is detected via a chemical protein detection method (e.g., via silver stain or another protein stain, such as Coomassie stain or a fluorescent protein stain).

c) Scale-Up

ABP early development is typically carried out on a small scale on a microarray slide or other similar scale using spots of micron-to-millimeter scale. For practical applications, polymers of micron size or larger can be useful for some uses, such as in protective powders or integrated into personal protective equipment such as face masks. Polymers for these microscale applications can be prepared by bulk or suspension polymerization. They can also be prepared by precipitation polymerization (Ye et al., 1999; Ye & Mosbach, 2001). Nanoscale polymer particles are preferred for in vivo (bloodstream) applications. Nanoparticles can also be superior to larger polymers for many ex vivo applications because of their high surface-to-volume ratios which provide more functional surface area per gram of polymer.

Large scale production of polymer nanoparticles can be carried out by emulsion polymerization (Odian, 1991; Rempp & Merrill, 1992).

### 4) In Vivo Use of ABPs as Anti-Viral Therapeutics or Protective Agents

Solutions of ABPs can be administered intravenously as is conventionally done with biological therapeutics such as antibodies. The dose (mg/kg) and frequency of administration can depend on several factors including efficacy, clearance rates, and toxicity. These parameters can be characterized according to established drug development and clinical testing procedures well known in the art of drug development.

Accordingly, in some embodiments, the presently disclosed subject matter provides a method of rendering a virus less infectious wherein the method comprises: identifying a biomimetic polymer that binds to a virus by performing screening (e.g., high throughput screening) of a polymer library against said virus or a protein component of said virus; formulating said virus-binding polymer; and introducing said polymer into an in vivo environment to bind to and sequester said virus, thus rendering said virus less infectious. In some embodiments, the in vivo environment is the circulating bloodstream of a human. In some embodiments, the virus is Ebola. In some embodiments, the virus is RSV. In some embodiments, the formulating comprises formulating the virus-binding polymer into a nanoscale or microscale antiviral binding polymer. In some embodiments, the preparation of antiviral biomimetic polymers against particular viruses followed by the in vivo administration of the ABPs (e.g., the nanoscale APGs) to a patient or subject generates a therapeutic response and/or assists in providing an improved immunological response. In some embodiments, introducing the virus-binding polymer into the in vivo environment prevents viral infection. In some embodiments, introducing the virus-binding polymer causes diminishment or cure of a viral disease (e.g., by reducing the amount of virus present in the in vivo environment or eliminating virus from the in vivo environment).

### 5) Ex Vivo Use of ABPs as Anti-Viral Prophylactic Measures

There are a number of applications for ABPs in an ex vivo environment. For example, in some embodiments, the ABPs can be used to capture viruses present in the air, on surfaces, or in fluids and thus prevent infection. ABPs can be used in many ways including: incorporated into medical masks (surgical, N95 or other), mixed into soap, lotion, or hand sanitizer, used as a powder on medical disposable gloves, added to wipes, tissues, or towels, or incorporated into other garments or wound treatment materials. For these applications, the format and size of the ABP particles can vary. ABPs can be nanoscale (<1000 nm) or microscale (>1000 nm) particles or a monolithic filter, or a membrane, among other materials formats.

Accordingly, in some embodiments, the presently disclosed subject matter provides a method of rendering a virus less infectious wherein the method comprises: providing a biomimetic polymer that binds a virus, optionally wherein providing a biomimetic polymer that binds a virus comprises identifying a biomimetic polymer that binds to a virus by performing screening (e.g., high throughput screening) of a polymer library against said virus or a protein component of said virus; formulating said virus-binding polymer; and introducing said polymer into an ex vivo environment to bind to and sequester said virus, thus rendering said virus less infectious. In some embodiments, the ex vivo environment is personal protective equipment selected from the group including, but not limited to, a surgical mask, a face mask, a hood, a helmet, goggles, boots, gloves, an apron, a shirt, pants, and a suit that covers the legs, torso and arms. In some embodiments, the ex vivo environment is a liquid, suspension, or emulsion selected from the group including, but not limited to, a body lotion, a face lotion, a hand lotion, a hand sanitizer, a liquid soap, an aerosol spray, and a mist spray. In some embodiments, the ex vivo environment is medical or other waste, or medical equipment selected from, but not limited to, a body bag, a disposable plastic waste bag, a sharps container, towels, waste water filter, and sponges. In some embodiments, the ex vivo environment is a filter for extracorporeal removal of virus from blood by double filtration plasmapheresis (DFPP), hemodialysis, or therapeutic apheresis. In some embodiments, the therapeutic apheresis is plasmapheresis, plateletpheresis, and leukapheresis. In some embodiments, the virus is Ebola. In some embodiments, the virus is RSV. In some embodiments, the formulating comprises formulating the virus-binding polymer into a nanoscale or microscale antiviral binding polymer.

In some embodiments, such as when the ABPs are introduced into personal protective equipment, medical waste, or when they are or into liquids, suspensions or emulsions, introducing the polymer into the ex vivo environment prevents viral infection. In some embodiments, such as when the ABPs are introduced into a filter for ex vivo treatment of a patient's removed blood in a extracorporeal device, the patient's removed and treated blood can be returned to the patient and the treated blood can experience a reduced viral load and as a result an decreased infection from the virus. This reduced viral load can also result in a therapeutic response (e.g., cure or diminishment of a viral infection) and/or provide an improved immunological response

### In Vivo Prophylactic Target: Zaire Ebola Virus (EBOV)

Zaire Ebola Virus (EBOV) is a filamentous pathogenic virus of the Filovidae family which causes hemorrhagic fever with high mortality in humans. Research has shown that administration of anti-Ebola antibodies to EBOV-infected rhesus macaques 24-48 hours post infection is effective in preventing disease or clinical symptoms (Olinger et al., 2012). This indicates that antibody-mediated clearance of EBOV takes place in the bloodstream of the infected individual. ABPs can be used as a low cost, effective antibody alternative in a recent infection treatment regimen or as a precaution in the case of possible imminent exposure. For example, ABPs can be administered in emergency infections such as soon after an accidental needle stick. Alternatively, anti-EBOV ABPs can be administered during a bioterror attack involving weaponized EBOV.

EBOV-binding ABPs are optimized to a size between 100 and 1000 nm diameter (nanoABPs) enabling viral binding and clearance from the bloodstream. The polymer is chosen based on ability to bind EBOV with high avidity. In particular, the nanoABP binds to EBOV capsid protein GP1,2 (Ascenzi et al., 2008). Other characteristics of the polymer include low toxicity, ability to clear EBOV causing viral inactivation, and long-lasting circulation in the absence of bound virus. These characteristics are determined by conventional drug studies of distribution, metabolism, excretion, toxicity, and optimal dosage, and pharmacokinetics.

Anti-EBOV ABPs are administered intravenously in saline-based solution by syringe at a dosage (mg/kg), frequency, and duration determined by the characterization studies described above.

### In Vivo Therapeutic Target: Zaire Ebola Virus (EBOV)

Anti-viral therapeutic ABPs can be developed for any pathogenic virus. There is a critical need for therapeutics to treat individuals with hemorrhagic fever caused by EBOV. Other than palliatives, there are currently no approved drugs for EBOV hemorrhagic fever. Ebola virus is also listed by the U.S. Centers for Disease Control and Prevention (CDC) as a top category (Category A) potential bioterrorism agent. Because catastrophic natural Ebola outbreaks and bioterror attacks can occur suddenly and without warning, it is important to have anti-EBOV treatments stockpiled and ready at all times. For this reason, nanoABPs are superior to natural antibody therapies which are costly to produce, require refrigeration, and have relatively short shelf lives.

As with prophylactic administration, for disease treatment, anti-EBOV nanoABPs are administered intravenously in saline-based solution by syringe at a dosage (mg/kg), frequency, and duration determined by conventional laboratory, animal, and clinical studies.

### Ex Vivo Therapeutic Target: Zaire Ebola Virus (EBOV)

The therapeutic effects of medicines in the bloodstream can be limited by short circulation lifetimes, harmful immune responses, or metabolite damage to organs such as the liver and kidneys. For these reasons, in some cases it can be advantageous to use ABPs therapeutically outside of the human body. An advantage of ABPs over many biological drugs such as antibodies is that they are physically and chemically stable and can be used in an extracorporeal manner, where blood removed from a patient can be filtered through an anti-virus ABP matrix and returned, virus-free, to the patient. For example, if an ABP is developed which binds to a pathogenic virus such as EBOV, but has therapeutically limiting in vivo characteristics, then the treatment can take place outside of the patient's body. For example, a normal hemodialysis machine (Fresenius, Baxter, Gambro, or others) fitted with an ABP-containing filter can be used in a hospital room. The filter can be a conventional semi-permeable membrane (made from cellulose triacetate, polysulfone, polymethylmethacrylate, or other polymeric material typically used in hemodialysis) embedded with anti-EBOV ABPs. The polymer particles can be larger than the membrane pore size to remain in the filter. The patient's whole blood can be removed, for example, via a central venous catheter to circulate through an ABP-containing filter to remove EBOV, and then returned to the patient. In hemodialysis, the flow rates are typically 200-400 mL/min such that complete dialysis (5000 mL blood) occurs every 15 minutes. The number of dialysis cycles and the frequency of treatment can depend on the patient's health and viral load.

### ABPs as Personal Protection Shielding: Zaire Ebola Virus (EBOV)

In some embodiments, ABP virus capture and removal can take place in a virus-susceptible environment such as a hospital or clinical laboratory to prevent particular viruses from infecting healthcare workers. For example, anti-EBOV ABPs can be incorporated into face masks, gloves and/or other personal protective garments. In another example, ABPs can be formulated as a protective powder on gloves. ABPs can also be formulated into an aerosol or mist spray to be used on surfaces and equipment. ABPs can also be formulated as an ingredient into soaps, lotions, and/or hand sanitizers to act as a topical skin barrier to EBOV.

In some embodiments, ABP protection can be near the mouth and nose, which are the most common entry points for pathogenic viruses. In the case of Ebola virus, the articles of personal protective equipment closest to the mouth and nose are face masks and gloves. Coating or incorporating anti-EBOV ABPs into masks and gloves captures EBOV which can be present in airborne or surface liquids, particles, or aerosols.

Face masks, including but not limited to surgical, dental, N95, or NIOSH masks, can be worn to protect the wearer from infection. Masks are manufactured by numerous companies, for example 3M and Honeywell, and manufacturing technology is well-established. Most masks function as a physical barrier, however Medline, Inc. (Mundelein, Ill.) sells a Curad Biomask, which the company claims inactivates 99.99% of tested flu viruses by the incorporation of the chemical agents citric acid, zinc, and copper.

In some embodiments, anti-EBOV containing face masks can be manufactured in a similar way to the Curad Biomask. The ABPs can replace the Curad Biomask chemicals. The outer surface of the masks is hydrophilic, which wicks any aqueous liquid such as saliva or blood into the interior of the mask where the ABPs are located. The ABPs can bind and sequester EBOV, preventing infection.

Anti-EBOV gloves can be made in at least two ways. Surgical or laboratory gloves are typically made from latex or nitrile. Historically, powder has been used as a lubricant in the manufacture of medical gloves to aid donning and to obviate gloves sticking together. At present, the more widely used dusting powders are cornstarch and calcium carbonate (CaCO₃). ABP powder can be added to or used in place of existing powders. Alternatively, in some embodiments, ABPs can be incorporated into gloves, similar to G-VIR antiviral gloves (G-VIR Glove, 2008) made by Hutchinson Sante (Paris, France). G-Vir medical gloves are composed of three layers: 2 mechanical layers of thermoelastic polymer (inner and outer layers) and a middle layer containing microdroplets of disinfectant solution. The disinfectant solution is a blend of chlorhexidine digluconate and dimethyl didecyl ammonium chloride salts, which are known to be active against enveloped viruses. Solutions of ABPs can be added to or used in place of existing disinfectant solutions in glove manufacturing.

Having now generally disclosed the subject matter, the same will be more readily understood through reference to the following examples, which are provided by way of illustration, and are not intended to be limiting of the presently disclosed subject matter, unless specified.

### EXAMPLES

### Example 1

### Preparation of a Polymer Microarray Library by In Situ Polymerization

Microarray slides of 2280 different ABP polymers can be fabricated in the combinations described above using 20 monomers and three cross-linkers. The slides can be used to screen for detection of binding to proteins, VLPs, or viruses as described below.

Slide preparation: Glass microscope slides (76×25 mm) are cleaned with 1.0 M NaOH and functionalized with 3-(trimethyoxysilyl)propyl methacrylate to provide a covalent anchor for the polymers (Hansen, 2014).

Monomers, cross-linker, and initiator: Twenty monomers are selected from the list above. Each combination of all possible pairs of monomers is polymerized with three cross-linkers (ethylene glycol dimethacrylate (EGDMA), N,N′-methylenebisacrylamide (MBA), and glycerol dimethacrylate). Each monomer-monomer-cross-linker combination is polymerized in four molar ratios; 1:1:1, 1:1:3, 1:3:1, and 3:1:1. Polymerization is initiated with tetramethylethylenediamine (TEMED) and 2,2′-azobis(2-isobutyronitrile) (AIBN).

Inkjet non-contact printing: A microarray of 2280 polymer spots (20×114 with each spot ˜200 μm in diameter) is prepared by printing monomers, crosslinkers, and initiators using a Microdrop Autodrop scientific inkjet printing platform fitted with an AD-K-501 piezo-driven Autodrop pipette (Microdrop Technologies GmbH, Germany) and allowing polymerization to take place under suitable conditions (Zhang et al., 2008; Hansen et al., 2014). Eight slides are printed for each screening experiment; four for quadruplicate binding of a target molecule/virion/VLP and four for quadruplicate of a control irrelevant molecule/virion/VLP.

### Example 2

### Preparation of a Polymer Microarray Library by Spotting Pre-Formed Polymers

As an alternative to performing on-microarray-slide polymerization, polymers are formed in advance and spotted on slides by contact printing. Polymers are formed as described (Khan et al., 2013; Duffy et al., 1014b).

To prepare a glass microscope slide, slides are cleaned, silanated, then dipped into a 2% solution of agarose at 65° C. (Khan et al., 2013; Duffy et al., 1014b).

Polymers are prepared and 1% (w/v) solutions in N-methyl-pyrrolidone (NMP) are printed from 384-well plates using a SpotBot 3 Personal Microarrayer (ArrayIt Corporation, Sunnyvale, Calif.). The printed slides are dried overnight in a vacuum at 40° C. before screening.

### Example 3

### High Throughput Screening of an ABP Microarray Library for M13 Bacteriophage-Binding ABPs

This example describes procedures for the discovery of ABPs that bind bacteriophage M13. M13 and the related fd phage are nonpathogenic filamentous viruses that infect E. coli bacteria and are well-established as laboratory tools, especially in the field of phage display of peptides and proteins. Phage M13 is used herein as a prototype virus to illustrate ABP discovery and development methods. These methods can be applied to dangerous and less accessible pathogenic filamentous viruses such as Ebola. Indeed, the aspect ratio and size of M13 (˜10 nm wide and ˜800 nm long) resembles that of filamentous Ebola virus (˜80 nm wide and ˜970 nm long) (FIG. 1).

Phage M13 can be obtained commercially, either as virus particles (New England Biolabs, Inc., Ipswich, Mass., Catalog #N0316S) or as a phage or phagemid vector (Antibody Design Laboratories, San Diego, Calif.). To obtain a sufficient mass of phage particles for screening and detection, phage particles are expressed in E. coli and secreted particles are purified by known methods such as precipitation in 20% PEG6000, 2.5 M NaCl, followed by centrifugation at 15,000 g (Engberg et al., 1995).

ABP microarrays can be prepared on a glass slide as described in Examples 1 and 2 above. For the M13 screen described in this example, the following monomers were used: 2-hydroxymethacrylate, N-isopropyl acrylamide, 2-methoxymethacrylate, acrylic acid, ethyl methacrylate, 2-di(ethylamino)ethylacrylate, butylmethacrylate, 2-di(methylamino)ethylacrylate, and 2-methoxyethylacrylate, in combinations of two or three different monomers per spot and varying percentages of each monomer, generating 380 polymers in total on a single microscope slide.

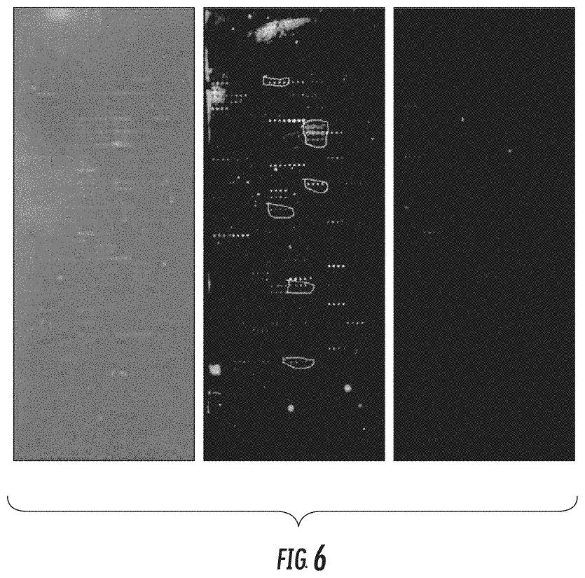

For screening, a phosphate-buffered saline/1.0% Tween (PBST) solution of phage particles was contacted with the microarray of ABPs for 3.5 hours. The M13 was at a concentration of 83 nanomolar (nM). After incubation the microarray was blocked in a mixture of 1% Bovine Serum Albumin and 0.1% Tween for one hour. The microarray slide was then incubated in the presence of the blocking mixture for two hours with a biotinylated anti-M13 antibody (GeneTex, Irvine, Calif.). This was followed by incubation with the fluorophore containing streptavidin-Cy5 (GE Healthcare Life Sciences, diluted 1000:1) for 30 minutes, also in the presence of the blocking mixture. After washing, the microarray on the slide was read on a fluorescent scanner (Axon GenePix 4000A) at 635 nm. Two controls were also carried out. The first was a microarray slide read directly in the fluorescent scanner for background fluorescence. The second was a microarray slide taken through all the reagent incubation and fluorescence scan steps but without any M13 phage present. See FIG. 6.

Continuing with reference to FIG. 6, ABP spots that fluoresce indicate the presence of bound M13 phage and hence M13-binding ABP. The results of the scan indicated that of the 380 polymers on the slide, 29 gave initial hits of which 8 polymers appeared to be M13 phage specific. The polymer compositions of the 8 hit polymers are provided below in Table 1, which indicates the number of monomers present in each positive hit and the relative percentages of the constituent polymers.

TABLE 1
Polymer compositions showing binding specificity for M13.

| Polymer | | | |
|---|---|---|---|
| Number | Monomer 1 (%) | Monomer 2 (%) | Monomer 3 (%) |
| PA44 | 2-hydroxyethyl | N-isopropyl- | — |
| | methacrylate | acrylamide | |
| | (70) | (30) | |
| PA235 | 2-hydroxyethyl | Acrylic acid | 2-di(ethyl-amino)ethyl |
| | methacrylate | (10) | |
| | (70) | | acrylate (20) |
| PA391 | ethyl | 2-di (ethyl- | — |
| | methacrylate | amino)ethyl | |
| | (70) | methacrylate | |
| | | (30) | |
| PA413 | butyl | 2-di (methyl- | — |
| | methacrylate | amino)ethyl | |
| | (50) | acrylate (50) | |
| PA430 | 2-methoxyethyl | 2-di(ethyl- | 2-methoxy- |
| | methacrylate | amino)ethyl | ethylacrylate |
| | (40) | methacrylate | (30) |
| | | (30) | |
| PA544 | 2-methoxyethyl | Acrylic acid | 2-di(ethyl- |
| | methacrylate | (10) | amino)ethyl |
| | (50) | | methyacrylate |
| | | | (40) |
| PA546 | 2-methoxyethyl | Acrylic acid | 2-di(ethyl- |
| | methacrylate | (15) | amino)ethyl |
| | (60) | | methacrylate |
| | | | (25) |
| PA561 | 2-methoxyethyl | Acrylic acid | 2-di(ethyl- |
| | methacrylate | (25) | amino)ethyl |
| | (50) | | methacrylate |
| | | | (25) |

The strength (avidity) of binding of positively identified polymer spots to M13 particles can be established by conventional methods known in the art, such as ELISA or Biacore (GE Healthcare Bio-Sciences, Piscataway, N.J., USA) measurements, while a degree of specificity can be demonstrated by measurement of M13 binding using similar methods well known in the art such as binding in the presence of a complex matrix such as whole blood, serum or plasma.

### Example 4

### Preparation of EBOV GP1,2 and High Throughput Screening for Binding to an ABP Microarray Library

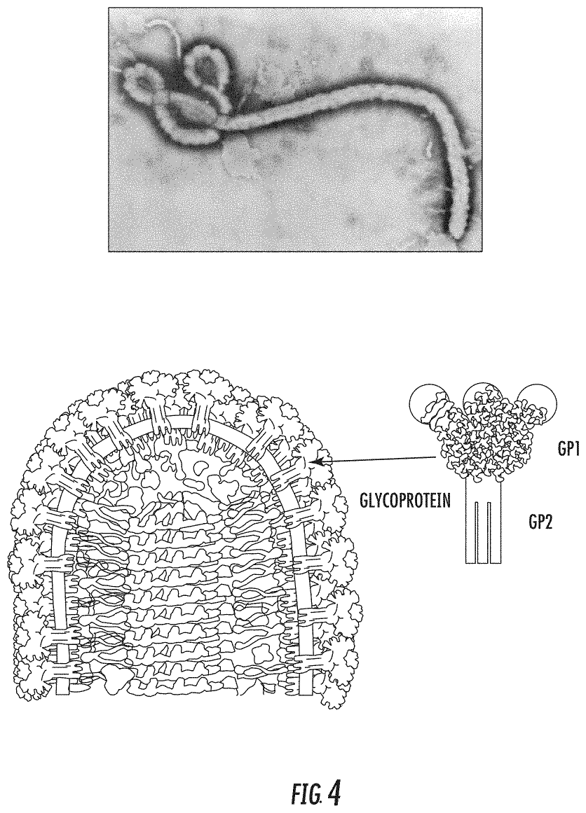

Therapeutics for Ebola Virus Disease are badly needed. ABPs can be discovered using whole Zaire Ebola virus, but a safer, more manageable strategy is to initially screen ABP libraries for binding to isolated capsid protein GP1,2 which is the sole surface protein coating Ebola. See FIG. 4. Whole and partial GP1,2 protein has been overexpressed and purified by a number of groups for protein structure/function studies including x-ray crystallography (Jeffers et al., 2002; Lee et al, 2008; Reynard et al., 2009; Dube et al., 2009).

Zaire ebolavirus (Mayinga strain) glycoprotein DNA is codon-optimized and the whole gene is synthesized (Blue Heron Biotech), then cloned into the pDISPLAY vector (Invitrogen, Carlsbad, Calif.) with a stop codon introduced before the internal transmembrane segment of the vector. GP1,2 is composed of more than 50% oligosaccharides by weight owing to a highly glycosylated mucin-like domain that is non-essential for cellular attachment or entry. To increase protein homogeneity and solubility, deletion of the mucin-like and transmembrane domains (residues 312-462 and 633-676) and mutations of two N-linked glycosylation sites (T42V and T230V) are created by overlap PCR and the QuikChange II site-directed mutagenesis kit, respectively. Large-scale expression of the T42V/T230V GP33-632Δmuc protein is performed using HEK293T cells transfected by standard calcium phosphate precipitation in 10-layer CellStack culture chambers (Corning, Corning, N.Y.). The DNA-calcium phosphate mixture is added to 70% confluent cells grown in DMEM plus 1× Pen/Strep and 5% (v/v) FBS. The supernatant is harvested four days post-transfection, concentrated using a Centramate tangential flow system (Pall Corp., Fort Washington, N.Y.), purified by anti-HA immunoaffinity chromatography and natively deglycosylated with peptide-N-glcosylase F (New England Biolabs, Ipswich, Mass.) in 1× PBS supplemented with 1.5 M urea at 37° C. (Dube et al., 2009).

Screening for binding of EBOV GP1,2 to ABPs is carried out on an ABP microarray library (Examples 1 and 2 describe library preparation.). To a microarray slide is added isolated GP1, 2 protein (0.1-10 micromolar) in a physiological solution such as phosphate-buffered saline (PBS) solution and allowed to incubate at room temperature for 60 minutes. Following three washes with PBS to remove unbound GP1,2, bound GP1,2 detected using biotinylated anti-GP1,2 antibody (IBT Bioservices affinity-purified rabbit polyclonal antibody, Cat #0301-015-BT or other anti-GP1,2 antibody) and streptavidin-DyLight conjugate (Pierce, Rockford, Ill.) binding followed by fluorescence scanning. Fluorescent spots indicate polymer library members that bind to GP1,2.

### Example 5

### Preparation of EBOV Virus-Like Particles for Screening

An alternative virus-free method to the discovery of anti-Ebola ABPs is through the use of Virus-Like Particles (VLP). VLPs are non-infectious synthetic particles with similar morphology and surface proteins as the viruses that they mimic. Methods have been established for preparation of Ebola-mimicking VLPs for laboratory use and as potential vaccines (Wool-Lewis & Bates, 1998; Warfield et al., 2003; Watanabe et al., 2004; Ou et al., 2012). These VLPs display on their surfaces GP1,2 which is the sole surface protein in Ebola virus and thus can be attractive multi-valent particles for the discovery and development of anti-Ebola ABPs.

Ebola VLPs are prepared by the method of Ou et al. (2012). VLPs made by this method lack the mucin-like domain, thus exposing potential ABP-protein binding regions. VLPs are produced by transient transfection of HEK 293T cells with two plasmids: one encoding Ebola virus GPΔMLD and the other encoding the gag-pol polyprotein precursor of the Moloney murine leukemia virus (MLV) core and enzymatic proteins (Ou et al. 2012). To maximize the yield of VLPs and their incorporation of GPΔMLD, the ratio of the two plasmids used for transfection is optimized to 2:1, gag-pol:env (Ou et al., 2012). After concentration and partial purification, the VLP preparation purity is confirmed with silver stained SDS-PAGE gels. The incorporation of GPΔMLD into the VLPs is confirmed by western blot analysis.

### Example 6

### Screening of ABP Libraries for Binding to Ebola Virus-Like Particles

To each of four identical polymer microarrays is added one of three different 300 μL of Ebola VLP solutions in phosphate-buffered saline (PBS). The Ebola VLP concentrations are 10, 100, and 1000 ng/mL. In parallel, three solutions (10, 100, and 1000 ng/mL) of control VLP particles bearing an irrelevant protein are also similarly incubated with four more identical polymer arrays. Thus, there are a total of 24 slides screened. The VLP/ABP microarrays are incubated on a slow platform shaker at room temperature for 60 minutes, then washed with PBS to remove unbound VLP. Bound target is then detected by immunodetection using a biotin-labeled polyclonal anti-Zaire Ebola antibody (IBT Bioservices, Gaithersburg, Md.) and streptavidin-DyLight conjugate (Pierce, Rockford, Ill.) binding followed by fluorescence scanning.

### Example 7

### Preparation of RSV VLP for Screening

Respiratory syncytial virus (RSV), which causes infection of the lungs and breathing passages, is a major cause of respiratory illness in young children (Empey et al., 2010; Lanari et al., 2013; Turner et al., 2014). Development of ABPs that bind RSV are useful as therapeutics and as ex vivo sequestering agents to prevent infections.

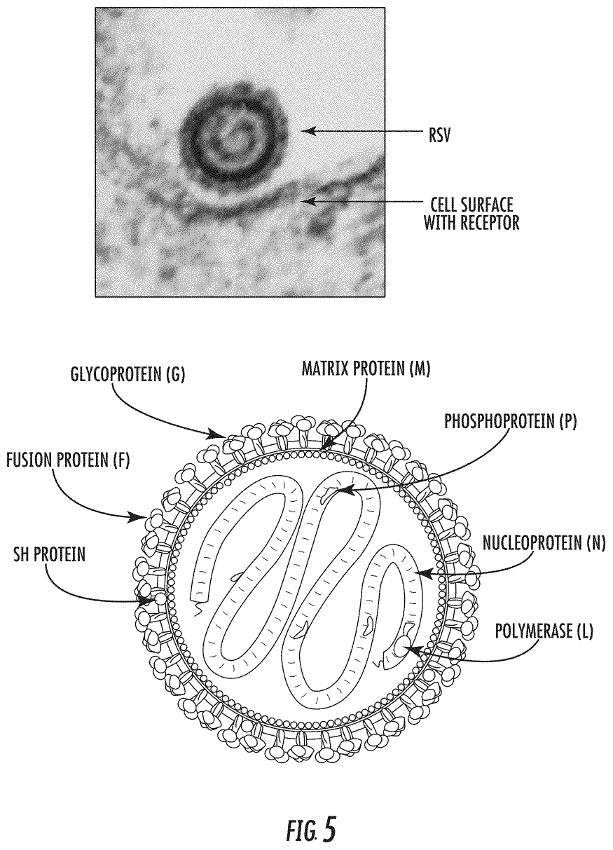

RSV is coated with two major proteins; Glycoprotein G (G) and Fusion protein F (F) (McLellan et al., 2013; Lanari et al., 2013). See FIG. 5. ABPs that bind one or both of these surface proteins can be expected to reduce infectivity. Protein F is an attractive target because there is less RSV strain-to-strain antigenic diversity than seen with Protein G. Protein G on the other hand is an attractive target because it also exists in the bloodstream in a soluble form thought to be an antibody decoy (Lanari et al., 2013).

RSV F- or G-protein surface bearing VLPs (RSV-F and RSV-G, respectively) have been previously described (Quan et al., 2011). These VLPs are useful for discovering and optimizing anti-RSV ABPs.

The preparation of RSV-G is described here using RSV G-protein gene from RSV strain A2 (Quan et al., 2011). Quan et al. also discloses a similar method for preparing RSV-F, which can alternatively be used.

Wild type RSV stocks are first prepared. HEP-2 cells are grown in tissue culture flasks in Dulbecco modified Eagle medium (DMEM) containing 10% fetal bovine serum (FBS). RSV is added, and virus adsorption is carried out in medium without serum for 1 hour at 37° C. with 5% CO₂. DMEM with 5% FBS is added to the flask and incubated for 2-3 days. RSV-infected cells are removed using a cell scraper and centrifuged at 3000 rpm for 30 minutes to remove supernatants. Infected cell pellets are sonicated and centrifuged at 4° C., and the supernatants are titrated by immunoplaque assay and stored at −80° C.

The RSV G gene is then polymerase chain reaction (PC) amplified from a cDNA clone of protein G by use of primers 5-AAAGAATTCACCATGTCCAAAAACAAGGACCAAC-3 (SEQ ID NO: 1) and 5-TTACTCGAGTACTGGCGTGGTGTGTTG-3 (SEQ ID NO: 2) and then cloned into pFastBac vector (Life technologies, Grand Island, N.Y.) via primer EcoRI/XhoI sites, resulting in plasmid pFastBac-G. RSV-G VLPs are produced by infecting Sf9 cells with rBVs expressing RSV-G. Cell culture supernatants are collected on day 2 post-infection with centrifugation at 6000 rpm for 20 minutes at 4° C. VLPs are concentrated with QuixStand (GE Healthcare, Piscataway, N.J.) and purified through a 20%-30%-60% discontinuous sucrose gradient at 30,000 rpm for 1 hour at 4° C. The VLP bands between 30% and 60% are collected and then diluted with phosphate-buffered saline (PBS) and pelleted at 28,000 rpm for 40 minutes at 4° C. VLPs are resuspended in PBS overnight at 4° C.

RSV-G VLPs are characterized by Western blots using polyclonal goat anti-RSV antibody used to probe RSV-G protein. Biotinylated antibody (AB19986, Abcam PLC, Cambridge, Mass.) binds to RSV-G, which is then detected using Streptavidin-HRP conjugate and color detection using the HRP substrate TMB.

### Example 8

### Screening of ABP Libraries for Binding to RSV

To each of four identical polymer microarrays is added one of three different 300 μL of RSV-G solutions in phosphate-buffered saline (PBS). The RSV-G concentrations are 10, 100, and 1000 ng/mL. In parallel, three solutions (10, 100, and 1000 ng/mL) of control VLP particles bearing an irrelevant protein are also similarly incubated with four more identical polymer arrays. Thus, there are a total of 24 slides screened. The RSV-G/ABP microarrays are incubated on a slow platform shaker at room temperature for 60 minutes, and then washed with PBS to remove unbound VLP. Bound RSV-G or control VLP is then detected by immunodetection using biotin-labeled polyclonal anti-RSV antibody (AB19986, Abcam PLC, Cambridge, Mass.), which binds to RSV-G. Bound antibody is then detected via streptavidin-DyLight conjugate (Pierce, Rockford, Ill.) binding followed by fluorescence scanning (e.g., Axon GenePix Personal 4100A).

### Example 9

### Preparation of ABP Nanoparticles by Emulsion Polymerization

Nanoscale polymers including ABPs can be prepared by emulsion polymerization, which is well known in the art (Odian, 1991; Rempp & Merrill, 1992). A monomer (for example methacrylic acid (MAA)) and a cross-linker (for example ethylene glycol dimethylacrylate (EDMA)) are mixed in water with an initiator in aqueous solution. The initiator can be one of a number initiators known in the art, including 1,1′-azobis(cyclohexanecarbonitrile) (AIBN), potassium or ammonium persulfate, hydrogen peroxide, and 2,2′-azobis(2-amidinopropane). A micelle-forming surfactant such as sodium palmitate is also used. Useful surfactants can also include other fatty acid soaps, sulfates, and sulfonates. The mixture is stirred at a speed to obtain the desired nanoparticle size. The ideal conditions can depend on the polymer to be prepared. Once prepared, ABPs can be purified and separated by size using centrifugation, filtration, and size exclusion chromatography.

### Example 10

### Preparation of ABP Nanoparticles from Solution Polymerization

Nanoscale polymers were prepared from solution polymerization forming a monolith. This example describes the general method which can be applied to many different polymers. A polymer was prepared by mixing in glass test tubes: monomer methacrylic acid (MAA, 0.884 mL), cross-linker ethylene glycol dimethylacrylate (EDMA, 10 mL), initiator 1,1′-azobis(cyclohexanecarbonitrile) (AIBN, 185 mg), and solvent chloroform (HPLC grade, 16 mL). The mixture was purged with argon for 4-5 minutes, then the tubes were sealed and irradiated with a handheld UV lamp at 4° C. overnight.

Solid polymer in test tubes was crushed and ground to a fine powder using a mortar and pestle. Powder in water was then wet sieved through a 25 micron sieve to remove large particles. The sieve filtrate was then filtered through a 0.45 micron syringe filter. Finally, the polymer nanoparticles were chromatographed on a Waters Styrogel HPLC size-exclusion column to acquire uniform nanopolymer particles of a nominal molecular weight of 100 kDa, which is similar in size to a typical protein molecule. The diameter of these nanoparticles was approximately 10 nm, but the described chromatography method is capable of producing particles in the 100-1000 nm range.

### Example 11

### In Vivo Administration of ABPs to a Human to Protect from Ebola Virus Disease

Delayed treatment of Ebola-infected rhesus macaques with monoclonal antibodies has shown that antibody protection is possible prior to the onset of Ebola Virus Disease symptoms (Olinger et al., 2012). Use of ABPs that similarly bind EBOV in vivo is expected to have the same biological protective effect.

The protective effect of ABPs in humans against EBOV depends on a number of variables which can be characterized by normal drug development methods including ADME/tox and pharmacokinetic studies. The optimum dose of ABPs depends in part on the binding affinity/avidity, the toxicity, and the bloodstream half-life.

If ABPs of similar therapeutic and biological characteristics to the Olinger et al. antibodies are used, then the treatment regimen will be similar. ABPs are administered in a physiologically appropriate solution prior to expected Ebola virus exposure or within approximately 48 hours post exposure. If a human is exposed to EBOV, then ABPs (for example, 50-100 mg/kg) are administered intravenously to reach a therapeutic dose. Additional doses of ABPs are administered whenever the bloodstream concentration of ABPs falls below the clinically-determined therapeutically effective dose, for example; every 12 hours, every 48 hours, or on days 4 and 8.

### Example 12

### In Vivo Administration of ABPs to a Human to Treat Ebola Virus Disease

ABPs can be used to treat a patient showing symptoms of Ebola Virus Disease. In this case, the exact dose and frequency of administration of therapeutic ABPs can depend on the results of in vivo studies in small animals, non-human primates, and humans. ABPs are administered intravenously in a physiologically appropriate solution to a patient at an optimum dose/frequency to reduce the viral load in that patient without severe toxicity. The dose is expected to be in the same range as natural antibody therapeutics (50-100 mg/kg) and the frequency of administration is expected to depend on the clinically-determined circulation half-life.

### Example 13

### In Vivo Administration of ABPs to Prevent RSV Disease

Synagis® (palivizumab, MedImmune LLC, Gaithersburg, Md.) is a monoclonal anti-RSV protein F IgG1 used as an antibody-boosting immuno-protectant in infants (Empey et al., 2010; Lanari et al., 2013; Turner et al., 2014). Synagis is not used therapeutically. The only approved therapeutic treatment for RSV is the small molecule RNA metabolism inhibitor, Ribavarin, which is costly and used in complicated cases (Turner et al., 2014). Anti-RSV ABPs could be used both for RSV protective and therapeutic uses. This example demonstrates how ABPs could be used in Synagis-like protection of infants against RSV infection.

The dose of anti-RSV (protein F or protein G) ABPs is similar to that for Synagis, which is 50 mg/mL injected in 1.0 mL physiological solution. ABPs (50-100 mg in 0.5-1.0 mL) can be administered by IV or IM injection. Synagis has a typical antibody bloodstream half-life of 20 days, which dictates the frequency of administration. Similarly, the half-life of ABPs in the bloodstream dictates its administration. The number of months duration of ABP administration is the same as that for Synagis.

### Example 14

### Incorporating ABPs into Lotions, Soaps, and Hand Sanitizers

Hand soaps often contain antimicrobial chemicals, the most common being Triclosan, that kill both beneficial and harmful bacteria, but have no effect against viruses. Anti-viral ABPs can be included into soaps and other skin products to protect individuals from infection with pathogenic viruses. Virus-binding nanoscale or microscale ABP particles can be blended into conventional healthcare products that are applied to the skin including lotions, liquid soaps, and hand sanitizers. In these cases, ABPs can be factory blended during the manufacturing process or they can be added and mixed in with packaged off-the-shelf commercial products. The effective concentration of ABPs in this application is in the nanogram-to-milligram per milliliter range. To be effective, the ABPs remain on the skin (especially with lotions and hand sanitizers) or bind and remove virus particles during washing (as in soaps). The overall effect is to reduce the infectiveness of virus particles.

### Example 15

### Extracorporeal Removal of Ebola Virus From Patients' Blood

Removal of virus particles from a patient's bloodstream by extracorporeal filtration of the blood can be an effective method to ameliorate viral disease. One company, Aethlon Medical (San Diego, Calif.), uses antibodies coupled to a polysulfone filter for specific removal of viruses. A drawback of this approach is that antibodies are relatively unstable molecules and generally require refrigeration. The replacement of antibody coupling with virus-binding microscale or nanoscale polymer can function similarly in the Aethlon apparatus, yet have much higher avidity for viruses and also much higher stability under room temperature storage conditions. The filters can be adapted to Aethlon manufacturing methods using nanoscale or microscale polymers by one skilled in the art. A patient with Ebola would experience a decline in blood viral load after dialysis with the polymer filters. The patient's blood can flow from one artery through a virus filtration cartridge (as with Aethlon Hemopurifier) and purified blood would return to the patient into another artery. Five liters of the patient's blood could flow through the filter in about 12 minutes (as per Aethlon) and several cycles could last a few hours until the blood viral load of Ebola is low or zero.

### REFERENCES

Arshady, R. & Mosbach, K. 1981. Macromol. Chem. Phys. 182, 687-692
Bolisay, L. D., Culver, J. N., & Kofinas, P. (2006) Biomaterials 27, 4165-8.
Bolisay, L. D., Culver, J. N., & Kofinas, P. (2007) Biomacromolecules 8, 3893-9.
Chiodo, F. et al. (2014) Beilstein J. Org. Chem. 10, 1339-46.
Dube, D. et al. (2009) J. Virol. 83, 2883-2891.
Duffy, C. R. et al. (2014a) Biomater. 35, 5998-6005.
Duffy, C. R. E. et al. (2014b) Biomater. Sci. 2, 1683-92.
Empey, K. M. et al. (2010) Clin. Infect. Dis. 50, 1258-1267.
G-VIR Glove. (2008) Health Technology Assessment Section, Medical Development Division, Ministry of Health, Malaysia.
Hansen, A. et al. (2014) Adv. Healthcare Mater. 3, 848-53.
Haupt, K. & Mosbach, K. (2000) Chem. Rev. 100, 2495-2504.
Hoshino, Y. et al. (2012) Proc. Natl. Acad. Sci., USA 109, 33-38.
Jeffers, S. A. et al. (2002) J. Virol. 76, 12463-72.
Jenik, M. et al. (2009) Anal. Chem. 81, 5320-5326.
Jones, K. L. et al. (2008) Nature 451, 990-993.
Khan, F. et al. (2013) Adv. Funct. Mater. 23, 2850-62.
Lanari, M. Et al. (2013) Clin. Dev. Immunol. 2013, doi: 10.1155/2013/359683.
Lee, J. E. et al. (2008) Nature 454, 177-182.
Liberski, A. et al. Chem. Commun. (2009a) 334-6.
Liberski, A. et al. J. Am. Lab. Autom. (2009b) 14, 285-93.
Macken, C. A. & Perelson, A. S. (1989) 86, 6191-6195.
Mathew, J. & Burchardt, O. (1995) Bioconjugate Chem. 6, 524-528.
Mathew-Krotz, J. et al. (1995) J. Am. Chem. Soc. 118, 8134-8135.
McLellan, J. S. et al. (2013) Curr. Top. Microbiol. Immunol. 372, 83-104.
Ngo, T. T. (1993) Molecular Interactions in Bioseparations, Plenum Press, New York.
Odian, G. (1991) Principles of Polymerization, pp. 301-303 and 335-355, 3^{rd} Ed., Wiley & Sons, New York.
Olinger et al. (2012) Proc. Natl. Acad. Sci. USA 109, 18030-18035.
Orlowski, P. et al. (2014) PLoS One 9(8): e104113.
Ou, W. et al. (2012) Virol. J. 9, 32.
Pernagallo, S. et al. (2011) J. Mater. Chem. 21, 96-101.
Quan, F.-S. et al. (2011) J. Infect. Dis. 204, 987-995.
Rempp, P. & Merrill, E. W. (1991) Polymer Synthesis, pp. 248-259, 2^{nd} Ed., Huthig & Wepf Verlag, New York.
Reynard, O. et al. (2009) J. Virol. 83, 9596-9601.
Rider, T. H. et al. (2011) PloS One 6(7): e22572.
Schillinger et al. (2012) Chem. Eur. J. 18, 14773-14783.
Shea, K. J. (1994) Trends Polymer Science 2, 166-173.
Shea, K. J. et al. (1993) J. Am. Chem. Soc. 115, 3368-3369.
Spivak, D. et al. (1997) J. Am. Chem. Soc. 119, 4388-4393.
Spivak, D. & Shea, K. J. (1998) Macromolecules 31, 2160-2165.
Turner, T. L. (2014) ClinicoEconomics and Outcomes Research 6, 2017-225.
Warfield, K. L. et al. (2003) Proc. Natl. Acad. Sci., USA 100, 15889-94.
Watanabe, S. et al. (2004) 78, 999.
Wool-Lewis, R. J. & Bates, P. (1998) J. Virol. 72, 3155-60.
Wulff, G. (1998) ChemTech, November, 19-26.
Wulff, G. & Sarhan, A. (1972) Angew. Chem., Intl. Ed. Engl. 11, 341
Ye, L. et al. (1999) Anal. Commun. 36, 35-38.
Ye, L. & Mosbach, K. (2001) React. Funct. Polym. 48, 149-157.
Yoshikawa, M. et al. (2001) Macromol. Mater. Eng. 286, 52-59.
Zeng, Z. et al. (2010) ACS Nano 4, 199.
Zhang, R. et al. (2008) Chem. Commun. 1317-1319.
Zhang, R. et al. (2013) Nat. Commun. 4, 1335.

All publications and patents mentioned in this specification are herein incorporated by reference to the same extent as if each individual publication or patent application was specifically and individually indicated to be incorporated by reference.

While the presently disclosed subject matter has been described in connection with specific embodiments thereof, it will be understood that it is capable of further modifications and this application is intended to cover any variations, uses, or adaptations of the subject matter following, in general, the principles of the subject matter and including such departures from the present disclosure as come within known or customary practice within the art to which the subject matter pertains and as may be applied to the essential features hereinbefore set forth.

1-26. (canceled)

27. A method of selecting a biomimetic, non-imprinted, non-biological polymer that has a specific binding specificity for a filamentous virus, the method comprising:
(a) preparing a library of non-imprinted, non-biological polymers;
(b) contacting said library with an aqueous solution containing a filamentous virus or a filamentous virus-like particle;
(c) detecting binding of said virus or virus-like particle to a subset of polymers within said library with a specific binding; and
(d) identifying at least one of the subset of polymers within said library which binds to said filamentous virus or virus-like particle with a specific binding specificity, thereby providing a biomimetic, non-imprinted non-biological polymer that has a specific binding specificity for a filamentous virus.

28. The method of claim 27, wherein the library of biomimetic, non-imprinted, non-biological polymers comprises at least 1000 different non-imprinted, non-biological polymers.

29. The method of claim 27, wherein the library of non-imprinted, non-biological polymers is arrayed on a microscope slide.

30. The method of claim 27, wherein the detecting is performed by immunoassay or chemical detection.

31. The method of claim 27, wherein the filamentous virus is a filovirus.

32. The method of claim 27, wherein the filamentous virus is a species of Ebola virus.

33. The method of claim 27, wherein the filamentous virus is Marburg virus.

34. The method of claim 27, wherein the filamentous virus is a bacteriophage.

35. The method of claim 27, wherein the filamentous virus is M13 bacteriophage.
